(19) United States
(12) Patent Application Publication
(10) Pub. No.: US 2023/0324418 A1
(43) Pub. Date: Oct. 12, 2023

(54) METHOD FOR DIAGNOSING AND TREATING COGNITIVE IMPAIRMENT AND USE THEREOF

(71) Applicant: BAO FENG BIOTECH (BEIJING) CO., LTD, Beijing (CN)

(72) Inventors: Xianyang CHEN, Beijing (CN); Wangting SONG, Beijing (CN); Jiarui HAN, Beijing (CN); Teng XUE, Beijing (CN)

(73) Assignee: BAO FENG BIOTECH (BEIJING) CO., LTD, Beijing (CN)

(21) Appl. No.: 18/021,211

(22) PCT Filed: Aug. 13, 2021
(86) PCT No.: PCT/CN2021/112405

§ 371 Date: Feb. 14, 2023

(30) Foreign Application Priority Data
Aug. 14, 2020 (CN) 202010816390.5

(51) Int. Cl. G01N 33/92

(52) U.S. Cl. CPC G01N 33/92; G01N 2405/08; G01N 2405/04; G01N 2405/02; G01N 2800/2814

(57) ABSTRACT

This disclosure provides a use of biomarker SM(d18:1/24:1(15Z)) in the preparation of a reagent or kit for diagnosing cognitive impairment. This disclosure also provides a method for diagnosing and treating cognitive impairment by detecting the content of biomarkers SM(d18:1/24:1(15Z)) and TG(16:0/18:0/18:4(6Z,9Z,12Z,15Z)), PC(P-16:0/22:4(7Z,10Z,13Z,16Z) or PC(P-18:0/18:4(6Z,9Z,12Z,15Z)) to determine whether the patient has cognitive impairment.

### FIELD

The invention belongs to the field of biological detection technology, in particular to a method for diagnosing and treating cognitive impairment and use thereof.

### BACKGROUND

It is well known that cognitive impairment is a progressive neurodegenerative disease. If not detected, paid attention to and intervened early, cognitive impairment is easy to develop into dementia, such as Alzheimer's disease (AD).

Senile plaques with extracellular beta-amyloid deposits and intracellular hyperphosphorylated tau nerve cell death throughout the brain of AD patients are major contributors to Alzheimer's disease, a disease that leads to progressive memory impairment, cognitive deficits, personality changes, and more. More than 5 million people worldwide suffer from Alzheimer's disease, and many more suffer from cognitive impairment.

Studies have shown that there are more than 10 million people with cognitive impairment, but so far, despite many studies into the pathogenesis, no good diagnostic markers have been found.

Mild cognitive impairment (MCI) is the intermediate state between normal cognition and AD. Studies show that mild cognitive impairment (MCI) is 10% to 20% prevalent in people over 65 years of age, and the cumulative probability of conversion to AD is 33%. Therefore, the early diagnosis and timely intervention of AD and pre-AD MCI will advance the prevention and treatment of AD disease, which is expected to effectively delay the progress of AD disease and reduce the burden on families, which is of great significance to the development of the whole society and medicine. Currently, the main methods of diagnosing mild cognitive impairment (MCD) and Alzheimer's disease (AD) include scale detection, imaging detection and cerebrospinal fluid biomarker detection. Scale detection requires questions and answers, which consumes a lot of medical resources and energy. Imaging detection requires MRI, PET and other expensive equipment which cannot be widely used. Cerebrospinal fluid sampling is traumatic and difficult, and patients and their families are reluctant to cooperate. Currently, there are no MCI and AD peripheral blood biomarkers with high accuracy and strong specificity.

Metabolomics is a new omics technology, which plays an increasingly important role in biological research, because it can reveal the unique chemical fingerprint characteristics of body cell metabolism. As an unbiased small molecule metabolite study method, metabolomics provides hope for the discovery of more biomarkers of cognitive impairment. A growing evidence suggests that neurological diseases are accompanied by disturbances in bile acids, fatty acids and amino acids. And these results suggest that metabolic disturbances may be predictive of cognitive impairment, but which substances can be detected as determination of cognitive impairment is unclear.

### SUMMARY

In order to effectively predict and diagnose cognitive impairment, the invention provides biomarkers for diagnosing cognitive impairment.

To achieve the above object, the invention adopts the following technical solution:

A biomarker for diagnosing cognitive impairment, SM(d18:1/24:1(15Z)). A use of the biomarker for diagnosing cognitive impairment in the preparation of test reagents as described above.

For use as described above, preferably, the biomarker also includes TG(16:0/18:0/18:4(6Z,9Z,12Z,15Z)), PC(P-16:0/22:4(7Z,10Z,13Z,16Z)) or PC(P-18:0/18:4(6Z,9Z,12Z,15Z)).

For the use described above, preferably, when the content units of SM(d18:1/24:1(15Z)) and TG(16:0/18:0/18:4(6Z,9Z,12Z,15Z) are mg/L, the TC value is calculated according to the formula TC=79.43−2.199×10⁻⁴×BM3+1.206×10⁻⁴×BM2, wherein BM3 is the content of SM(d18:1/24:1 (15Z)), and BM2 is the content of TG(16:0/18:0/18:4(6Z,9Z,12Z,15Z)). Cognitive impairment is predicted according to TC value: if TC≥0.5, it is determined to be cognitive impairment, and if TC<0.5, it is determined to be normal.

For the use described above, preferably, when the content units of SM(d18:1/24:1(15Z)) and PC(P-16:0/22:4(7Z,10Z,13Z,16Z) are mg/L, the TC value is calculated according to the formula 604.8−5.028×10⁻⁴×BM3−1.909×10⁻³×BM4, wherein BM3 is SM(d18:1/24:1(15Z)) and BM4 is PC(P-16:0/22:4(7Z,10Z,13Z,16Z)). Cognitive impairment was predicted according to TC value: if TC≥0.421, it is determined to be cognitive impairment and if TC <0.421, it is determined to be normal.

For the use described above, preferably, when the content units of SM(d18:1/24:1(15Z)) and PC(P-18:0/18:4(6Z,9Z,12Z,15Z) are mg/L, the TC value is calculated according to the formula: TC=48.98−4.266×10⁻⁵×BM3−1.897×10⁻⁴×BM5, wherein BM3 is SM(d18:1/24:1(15Z)), and BM5 is PC(P-18:0/18:4(6Z,9Z,12Z,15Z)). Cognitive impairment is predicted according to TC value: if TC≥0.749, it is determined to be cognitive impairment and if TC<0.749, it is determined to be normal.

The invention further provides a method for diagnosing cognitive impairment, comprising detecting the content of SM(d18:1/24:1(15Z)) in the serum of a subject, and the content of any of TG(16:0/18:0/18:4(6Z,9Z,12Z,15Z)), PC(P-16:0/22:4(7Z,10Z,13Z,16Z)) or PC(P-18:0/18:4(6Z,9Z,12Z,15Z)), and calculating the TC value to determine whether the subject has cognitive impairment.

For the method described above, preferably, the content of SM(d18:1/24:1(15Z)) measured is denoted as BM3, and the content of TG(16:0/18:0/18:4(6Z,9Z,12Z,15Z)) measured is BM2, TC value is calculated according to the formula TC=79.43−2.199×10⁻⁴×BM3+1.206×10⁻⁴×BM2, if TC≥0.5, it is determined to be cognitive impairment, if TC<0.5, it is determined to be normal.

For the method described above, preferably, the content of SM(d18:1/24:1(15Z)) measured is denoted as BM3, and the content of PC(P-16:0/22:4(7Z,10Z,13Z,16Z) measured is denoted as BM4, TC value is calculated according to the formula TC=604.8−5.028×10⁻⁴×BM3−1.909×10⁻³×BM4, if TC≥0.421, it is determined to be cognitive impairment, if TC<0.421, it is determined to be normal.

For the method described above, preferably, the content of SM(d18:1/24:1(15Z)) measured is denoted as BM3, and the content of PC(P-18:0/18:4(6Z,9Z,12Z,15Z)) measured is denoted as BMS, TC value is calculated according to the formula TC=48.98−4.266×10⁻⁵×BM3−1.897×10⁻⁴×BM5, if TC≥0.749, it is determined to be cognitive impairment, if TC<0.749, it is determined to be normal.

For the method described above, preferably, ultra-high performance liquid chromatography-mass spectrometry is used.

Further, preferably, the detection conditions of ultra-high performance liquid chromatochromatoid-mass spectrometry are C18 chromatographic column, mobile phase: 10 mM ammonium formate—0.1% formic acid-acetonitrile as phase A and 10 mM ammonium formate—0.1% formic acid-isopropanol-acetonitrile as phase B, ion source temperature: 120° C., dissolution temperature: 600° C., gas flow rate: 1000 L/h, flowing gas:nitrogen, capillary voltage: 2.0 kV(+)/cone voltage: 1.5 kV(−), and cone voltage: 30V.

A method for treating cognitive impairment, comprising: (a) diagnosing the patient with cognitive impairment using the method according to any of claims 1 to 5, (b) treating the diagnosed patient with cognitive impairment medication, and (c) diagnosing the rehabilitation condition by the method according to any of claims 1 to 5.

A test kit for diagnosing cognitive impairment, comprising standard SM(d18:1/24:1(15Z)), and any one of TG(16:0/18:0/18:4(6Z,9Z,12Z,15Z)), PC(P-16:0/22:4(7Z,10Z,13Z,16Z)) or PC(P-18:0/18:4(6Z,9Z,12Z,15Z)), mobile phase A: containing 10 mM ammonium formate and 0.1% formic acid as solute, and acetonitrile and water with a volume ratio of 60:40: water as solvent; mobile phase B: containing 10 mM ammonium formate and 0.1% formic acid as solute, and isopropyl alcohol and acetonitrile with volume ratio of 90:10 as solvent.

The invention brings the following beneficial effects:

The present invention provides a biomarker for diagnosing cognitive impairment, SM(d18:1/24:1(15Z)) in combination with TG(16:0/18:0/18:4(6Z,9Z,12Z,15Z)), PC(P-16:0/22:4(7Z,10Z,13Z,16Z)) or PC(P-18:0/18:4(6Z,9Z,12Z,15Z)) is detected for its content to predict cognitive impairment according to TC value, helping diagnosing whether the subject has a predisposition to cognitive impairment and can be used for prevention in advance.

### DESCRIPTION OF DRAWINGS

FIG. 1 shows VIP>1 samples in positive and negative ion mode.

FIG. 2 shows the score plot of (O)PLS-DA in positive and negative ion mode.

FIG. 3 shows the S-plot in positive and negative ion mode.

FIG. 4 shows the ROC curve based on logistic regression model (variable BM3+BM2).

FIG. 5 shows the ROC curve based on logistic regression model (variable is BM3+BM4).

FIG. 6 shows ROC curve based on logistic regression model (variable is BM3+BM5).

### DETAILED DESCRIPTION

The following examples are used to further illustrate the invention, but shall not be construed as a limitation of the invention. Without deviating from the spirit and essence of the invention, any modification or substitution of the invention shall fall within the scope of the invention.

Unless otherwise specified, the technical means used in the examples are conventional means known to the person skilled in the field.

### EXAMPLE 1

### I. Patient Group Standard

1. Sample Group for Model Establishment of 80 People (Internal Group, Which Refers to the Sample Group Used in the Establishment of Prediction Model)

Control group: 1:1 male to female ratio, age range: over 45 years old, MMSE scale score>26, Moca scale score>27, and no abnormality shown by MRI.

Patient group: male to female ratio: 1:1, age range: over 45 years old, MMSE scale score>26, Moca scale score>27, and partial abnormality shown by MRI.

2. Sample Group for Model Verification of 80 People (External Group, that is, the Sample Used for Model Verification (Non-internal Group)), the Sampling Standard was the Same as Above.

### II. Experimental Instruments and Reagents

Sample collection: serum from patients clinically assessed as normal and cognitively impaired was selected for the experiment.

1. Refrigerated centrifuge: Model D3024R, Scilogex Corporation, USA; 2. Vortex Oscillator: Model MX-S, Scilogex Corporation, USA; 3. High resolution Mass spectrometer: ESI-QTOF/MS; Model: Xevo G2-S Q-TOF; Manufacturer: Waters, Manchester, UK; 4. Ultra-high performance liquid chromatography: UPLC; Model: ACQUITY UPLC I-Class System; Manufacturer: Waters, Manchester, UK; 5. Data acquisition software: MassLynx4.1; Manufacturer: Waters; 6. Analysis and identification software: Progenesis QI; Manufacturer: Waters.

Experimental reagents: isopropyl alcohol, acetonitrile, formic acid, ammonia formate, leucine enkephalin, sodium formate; manufacturer: Fisher.

### III. Experimental Methods

1. Sample Pretreatment

The collected serum samples were thawed on ice, 200 μL plasma was extracted with 600 μL pre-cooled isopropyl alcohol, vortexed for 1 min, and incubated at room temperature for 10 min. Then the extraction mixture was stored overnight at −20° C., centrifuged at 4000 r for 20 min, and the supernatant was transferred to a new centrifuge tube, diluted with isopropyl alcohol/acetonitrile/water (volume ratio of 2:1:1) to 1:10. The samples were stored at −80° C. before LC-MS analysis. In addition, 10 μL of each extraction mixture was combined to prepare mixed plasma samples.

2. Ultra High Performance Liquid Chromatography-mass Spectrometry Method for Lipidomics

The samples were analyzed using ACQUITY UPLC(Waters, USA) connected to the Xevo-G2XS High Resolution Time of Flight (QTOF) Mass spectrometer (Waters) with ESI. CQUITY UPLC BEH C18 column (2.1×100 mm, 1.7 μm, Waters), and mobile phase consisted of 10 mM ammonium formate—0.1% formic acid-acetonitrile (A, acetonitrile:water=60:40, v/v) and 10 mM ammonium formate—0.1% formic acid-isopropyl alcohol-acetonitrile (B, isopropyl alcohol:acetonitrile=90:10, v/v) were used. Pilot trials with 10-, 15-, and 20-minute washout periods were conducted prior to the large-scale study to assess the potential impact of mobile phase composition and flow rate on lipid retention time. In the positive ion mode (PIM), rich lipid precursor ions and fragments separated in the same order, with similar peak shapes and ionic strengths. In addition, the mixed quality control (QC) sample with a 10-minute washout period also exhibited basal peak strength of precursor and fragment similar to the test sample. The flow rate of mobile phase was 0.4 mL/min. The column was initially eluted with 40% B, 43% B in 2 min according to linear gradient, and 50% B within 0.1 min, 54% B in 3.9 minutes according to linear gradient, and 70% B within 0.1 min. In the last part of the gradient, the amount of B increased to 99% in 1.9 minutes. Finally, solution B was returned to 40% within 0.1 min and the column was balanced for 1.9 min before the next sample injection. The lipids were detected by Xevo-G2XS QTOF mass spectrometer with a sample size of 5 μL/time, collection range of m/z 50˜1200 years, and collection time of 0.2 s/time. The ion source temperature was 120° C., the desolutizing temperature was 600° C., the gas flow rate was 1000 L/h, and nitrogen was used as the flowing gas. The capillary voltage was 2.0 kV(+)/the cone voltage was 1.5 kV(−), and the cone voltage was 30V. Sodium formate and leucine enkephalin were used as correction solution (coming with waters Mass spectrometer). The samples were sorted randomly. A quality control (QC) sample was injected into every 10 samples and analyzed to investigate repeatability of the data.

### IV. Result Analysis

1. Multivariate Statistics was Used to Search for Substances with Serum Differences

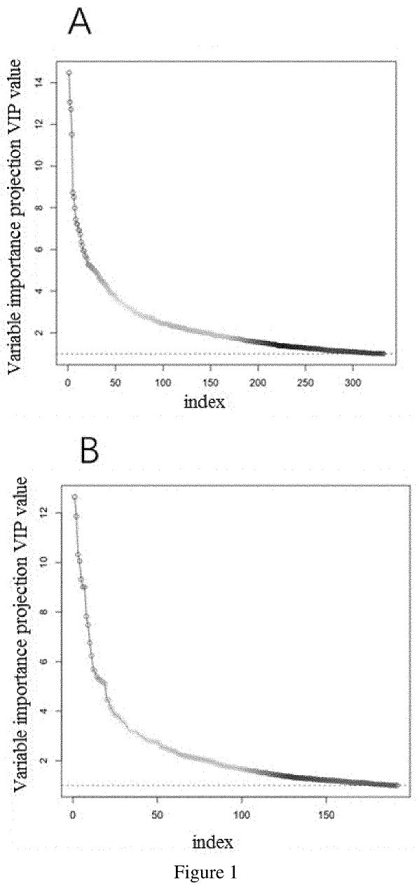

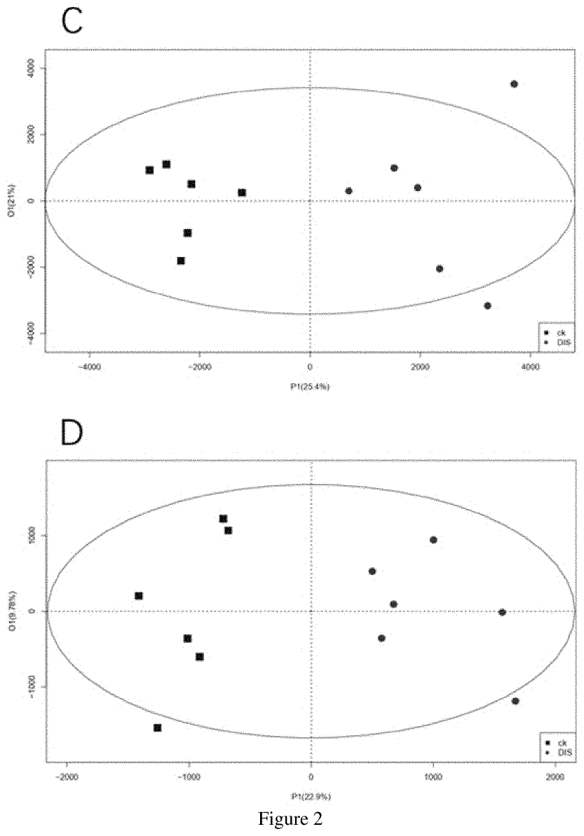

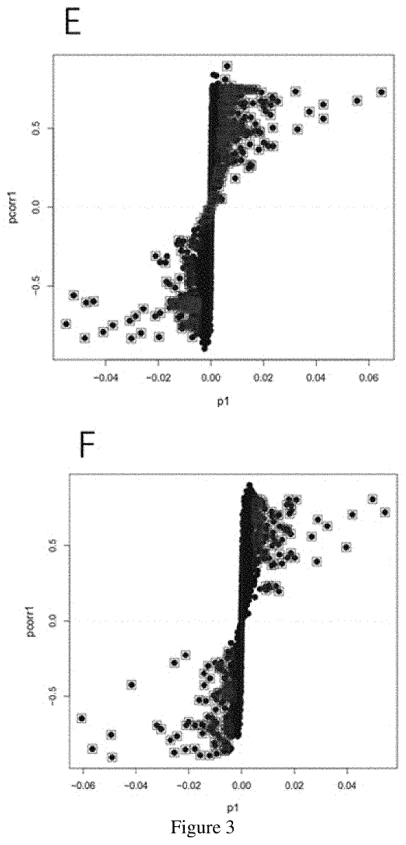

For the internal group, orthogonal partial least squares discriminant analysis (OPLS-DA) combined with orthogonal signal correction (OSC) and PLS-DA methods were used to screen differential variables by removing uncorrelated differences. FIG. 1 shows the metabolite of VIP>1 in positive and negative ion mode. VIP value was the variable importance projection of the first principal component of OPLSDA, usually VIP>1 was the commonly used evaluation criteria for metabolomics, as one of the criteria for screening differential metabolites, in which A was in positive ion mode, B was in negative ion mode; FIG. 2 shows the score plot of (O)PLS-DA in the positive and negative ion mode, wherein C was the score plot of (O)PLS-DA in the positive ion mode, and D was the score plot of (O)PLS-DA in the negative ion mode, in the score plot obtained by the first principal component and the second principal component in cognitive impairment group (DIS) and blank control group (CK) through the way of dimensionality reduction, the horizontal coordinate represented the difference between the groups, the vertical coordinate represented the difference within the group, and the two groups of results separation was good, indicating that this solution can be used. FIG. 3 shows the S-plot in positive and negative ion mode, E was the S-plot in positive ion mode, and F was the S-plot in negative ion mode. The horizontal coordinate represented the co-correlation coefficient between principal component and metabolite, and the vertical coordinate represented the correlation coefficient between principal component and metabolite, under the condition satisfying p<0.05 and VIP>1 at the same time, there were 59 differences in the negative ion mode and 117 differences in the positive ion mode.

2. Jorden Index Analysis

In order to further narrow the scope, the VIP threshold was raised to 3, while reflecting that the difference in fold between normal and patients was less than 0.8 times, or more than 1.2 times, and 10 compounds were finally obtained. See Table 1 for details.

Then, they were calculated by youden Yoden index to reflect the diagnostic and predictive effect of a single indicator on the whole. The results were shown in Table 1 below:

TABLE 1
Analysis of the Yoden index of lipids
associated with cognitive impairment

| Name of compounds | AUC | specificity | sensitivity |
|---|---|---|---|
| TG(16:0/18:1(9Z)/18:2(9Z,12Z)) | 0.861 | 0.833 | 0.833 |
| PC(22:6(4Z,7Z,10Z,13Z,16Z,19Z)/18:0) | 0.889 | 0.833 | 0.833 |
| SM(d18:1/24:1(15Z)) | 0.917 | 0.833 | 1 |
| TG(16:0/18:1(9Z)/18:3(6Z,9Z,12Z)) | 0.917 | 1 | 0.833 |
| PC(O-16:0/20:4(8Z,11Z,14Z,17Z)) | 0.861 | 0.833 | 0.833 |
| SM(d18:2/24:1) | 0.944 | 0.833 | 1 |
| SM(d16:1/20:0) | 0.889 | 1 | 0.667 |
| PC(P-16:0/22:4(7Z,10Z,13Z,16Z)) | 0.917 | 0.833 | 1 |
| PE(18:0/17:0) | 0.889 | 0.667 | 1 |
| PC(P-18:0/18:4(6Z,9Z,12Z,15Z)) | 0.917 | 0.833 | 1 |

Table 1 showed the area under the curve (AUC), sensitivity and specificity of individual metabolites in predicting cognitive impairment. Among the above 20 lipids, SM(d18:2/24:1) was the best in prediction (AUC=0.944). And 24:1 indicated the presence of a neuric acid chain.

3. 70% Cross-verification Results for Internal Group

According to the above results, variable compounds with YOUDEN AUC greater than 0.9 were selected for the next analysis.

TABLE 2

| No. | m/z | Name of compounds | AUC |
|---|---|---|---|
| BM1 | 855.6603 | SM(d18:2/24:1) | 0.944 |
| BM2 | 896.770388 | TG(16:0/18:0/18:4(6Z,9Z,12Z,15Z)) | 0.917 |
| BM3 | 813.6863083 | SM(d18:1/24:1(15Z)) | 0.917 |
| BM4 | 838.5974778 | PC(P-16:0/22:4(7Z,10Z,13Z,16Z)) | 0.917 |
| BM5 | 810.5649318 | PC(P-18:0/18:4(6Z,9Z,12Z,15Z)) | 0.917 |

The internal group was randomly divided into 7 groups. One group was selected as the verification set and the others as the training set. This process was repeated seven times to investigate the optimal combination of variables. The results including AUC, sensitivity and specificity, were averaged and calculated with statistical significance, as shown in Table 3.

TABLE 3

| | logistic | | |
|---|---|---|---|
| | regression | | |
| Combination | AUC | sensitivity | specificity |
| BM3 + BM2 | 1 | 1 | 1 |
| BM3 + BM4 | 1 | 1 | 1 |
| BM3 + BM5 | 0.972 | 1 | 1 |

AUC values were not significant (p<0.05) between combinations.

Based on the above, logistic regression models A, B and C were established as follows:

The variable of “Model A” was BM3+BM2, the TC value was calculated according to formula: TC=79.43−2.199×10⁻⁴×BM3+1.206×10⁻⁴BM2, wherein BM3 was the content of SM(d18:1/24:1(15Z)), BM2 was the content of TG(16:0/18:0/18:4(6Z,9Z,12Z,15Z)), and cognitive impairment was predicted according to TC value: if TC≥0.5, it was determined to be cognitive impairment; and if TC<0.5, it was determined to be normal.

The variable of “Model B” was BM3+BM4, and the TC value was calculated according to formula: TC=604.8×5.028×10⁻⁴×BM3−1.909×10⁻³ ×BM4, wherein, BM3 was the content of SM(d18:1/24:1(15Z)), BM4 was the content of PC(P-16:0/22:4(7Z,10Z,13Z,16Z)), and cognitive impairment was predicted according to TC value: if TC>0.421, it was determined to be cognitive impairment, and if the TC<0.421, it was determined to be normal.

The variable of “Model C” was BM3+BM5, TG(13:0/17:1(9Z)/20:2(11Z,14Z))+SM(d18:1/24:1(15Z)). the TC value was calculated according to formula: 48.98−4.266×10⁻⁵BM3×1.897×10⁻⁴×BM5, wherein, BM3 was the content of SM(d18:1/24:1(15Z)), BM5 was the content of PC(P-18:0/18:4(6Z,9Z,12Z,15Z)), and cognitive impairment was predicted according to TC value: if TC≥0.749, it was determined to be cognitive impairment, and if TC<0.749, it was determined to be normal.

4. External Data Set and Logistic Regression Model Verification

The accuracy of the above results was verified by the data set of external group, and the corresponding ROC curve was drawn. The results were as follows:

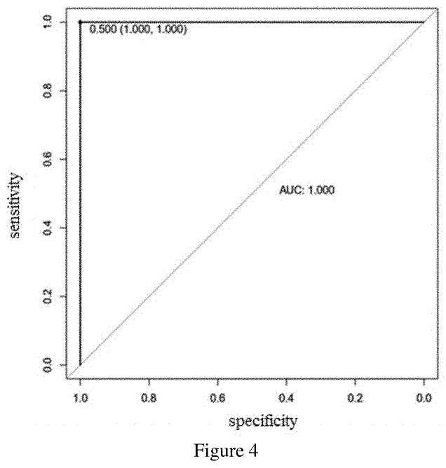

The variable of model A was BM3+BM2, the results are shown in FIG. 4, Sensitivity=1, Specificity=1, Accuracy=1.

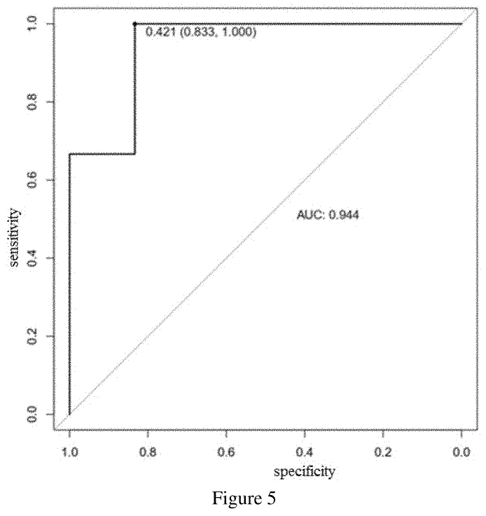

The variable of model B were BM3+BM4, the results are shown in FIG. 5, Sensitivity=1, Specificity=0.833, Accuracy=1.

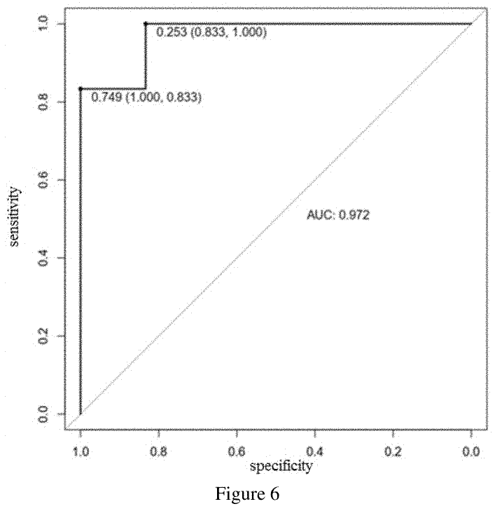

The variable of “Model C” was BM3+BM5, TG(13:0/17:1 (9Z)/20:2(11Z,14Z))+SM(d18:1/24:1 (15Z)). The results are shown in FIG. 6, Sensitivity=1, Specificity=0.833, Accuracy=1.

The data showed: SM (d18:1/24:1 (z) 15), or combinations with TG(16:0/18:0/18:4(6Z,9Z,12Z,15Z)), PC(P-16:0/22:4(7Z,10Z,13Z,16Z)), or PC(P-18:0/18:4(6Z,9Z,12Z,15Z)) all demonstrated very high diagnostic capabilities and were capable of clinical kit applications.

Comparative analysis of sample information showed that BM1, BM3, BM4 and BM5 of the above five biomarkers showed a downward trend in the cognitive impairment group compared with the normal group, while BM2 showed the opposite trend.

### EXAMPLE 2

A test kit for diagnosing cognitive impairment comprised standard SM(d18:1/24:1(15Z)), and any one of TG(16:0/18:0/18:4(6Z,9Z,12Z,15Z)), PC(P-16:0/22:4(7Z,10Z,13Z,16Z)) or PC(P-18:0/18:4(6Z,9Z,12Z,15Z)), mobile phase A containing 10 mM ammonium formate and 0.1% formic acid as solute, and acetonitrile and water with a volume ratio of 60:40 as solvent; and mobile phase B containing 10 dmM ammonium formate and 0.1% formic acid as solute, isopropyl alcohol and acetonitrile with volume ratio of 90:10 as solvent. wherein, 10 mM ammonium formate—0.1% formic acid-acetonitrile (A, acetonitrile: water=60:40, v/v) was prepared by dissolving 0.63 g ammonium formate and 10 g formic acid with acetonitrile-water solution (acetonitrile:water=60:40, v/v) to make total volume of 1000 mL.

10 mM ammonium formate—0.1% formic acid-isopropyl alcohol-acetonitrile (B, isopropyl alcohol:acetonitrile=90:10, v/v) was prepared by dissolving 0.63 g ammonium formate, 10 g formic acid with isopropyl alcohol-acetonitrile solution (isopropyl alcohol:acetonitrile=90:10, v/v) to make total volume of 1000 mL.

For sample detection, sample pretreatment and ultra-high performance liquid chromatography-mass spectrometry in Example 1 were used. Meanwhile, the standard SM(d18:1/24:1(15Z), and TG(16:0/18:0/18:4(6Z,9Z,12Z,15Z)), PC(P-16:0/22:4(7Z,10Z,13Z,16Z)) or PC(P-18:0/18:4(6Z,9Z,12Z,15Z)) were used as reference for detection.

### EXAMPLE 3

A method for treating cognitive impairment included: (a) the kit and detection method as shown in Example 2 were used to diagnose patients with cognitive impairment, (b) the content of SM(d18:1/24:1(15Z)) was denoted as BM3, and the content of TG(16:0/18:0/18:4(6Z,9Z,12Z,15Z)) was BM2, TC value was calculated according to the formula TC=79.43−2.199×10⁻⁴×BM3+1.206×10⁻⁴×BM2, if TC≥0.5; or the content of PC(P-16:0/22:4(7Z,10Z,13Z,16Z)) was denoted as BM4, and the TC value was calculated according to the formula TC=604.8−5.028×10⁻⁴×BM3−1.909×10⁻³×BM4, if TC≥0.421; or the content of PC(P-18:0/18:4(6Z,9Z,12Z,15Z)) measured was denoted as BM5, and the TC value was calculated according to the formula TC=48.98−4.266×10⁻⁵×BM3−1.897×10⁻⁴×BM5, if TC≥0.749; 10 patients were considered to be suffering from cognitive impairment, which was confirmed by the above method and further confirmed by MRI test. After 2 months of treatment with Memantine drug according to the conventional method, screening and determination by the above method showed that the patients' condition was improved and controlled. This was consistent with the confirmed results of MRI nuclear magnetic detection, showing that the screening method of the invention was as accurate as the confirmed results of nuclear magnetic diagnosis, indicating that the diagnostic method of the invention was accurate and reliable.

1. A method for detecting cognitive impairment, comprising detecting the content of SM(d18:1/24:1(15Z)) in the serum of a subject, and the content of any of TG(16:0/18:0/18:4(6Z,9Z,12Z,15Z)), PC(P-16:0/22:4(7Z,10Z,13Z,16Z)) or PC(P-18:0/18:4(6Z,9Z,12Z,15Z)), and calculating the TC value to determine whether the subject has cognitive impairment.

2. The method according to claim 1, wherein the content of SM(d18:1/24:1(15Z)) measured is denoted as BM3, and the content of TG(16:0/18:0/18:4(6Z,9Z,12Z,15Z)) measured is BM2, TC value is calculated according to the formula TC=79.43−2.199×10⁻⁴×BM3+1.206×10⁻⁴×BM2, if TC≥0.5, it is determined to be cognitive impairment, if TC<0.5, it is determined to be normal.

3. The method according to claim 1, wherein the content of SM(d18:1/24:1(15Z)) measured is denoted as BM3, and the content of PC(P-16:0/22:4(7Z,10Z,13Z,16Z) measured is denoted as BM4, TC value is calculated according to the formula TC=604.8−5.028×10⁻⁴×BM3−1.909×10⁻³×BM4, if TC≥0.421, it is determined to be cognitive impairment, if TC<0.421, it is determined to be normal.

4. The method according to claim 1, wherein the content of SM(d18:1/24:1(15Z)) measured is denoted as BM3, and the content of PC(P-18:0/18:4(6Z,9Z,12Z,15Z)) measured is denoted as BMS, TC value is calculated according to the formula TC=48.98−4.266×10⁻⁵×BM3−1.897×10⁻⁴×BM5, if TC≥0.749, it is determined to be cognitive impairment, if TC<0.749, it is determined to be normal.

5. The method according to claim 1, using ultra-high performance liquid chromatography-mass spectrometry.

6. A method for treating cognitive impairment, comprising:
(a) diagnosing the patient with cognitive impairment,
(b) treating the diagnosed patient with cognitive impairment medication, and
(c) diagnosing the rehabilitation condition,
wherein the diagnosing the patient with the cognitive impairment or the diagnosing the rehabilitation condition is implemented by the method according to claim 1.

7. A test kit for diagnosing cognitive impairment, comprising standard SM(d18:1/24:1(15Z)), and any one of TG(16:0/18:0/18:4(6Z,9Z,12Z,15Z)), PC(P-16:0/22:4(7Z,10Z,13Z,16Z)) or PC(P-18:0/18:4(6Z,9Z,12Z,15Z)), mobile phase A: containing 10 mM ammonium formate and 0.1% formic acid as solute, and acetonitrile and water with a volume ratio of 60:40: water as solvent; mobile phase B: containing 10 mM ammonium formate and 0.1% formic acid as solute, and isopropyl alcohol and acetonitrile with volume ratio of 90:10 as solvent.
